(19) United States
(12) Patent Application Publication
(10) Pub. No.: US 2009/0281592 A1
(43) Pub. Date: Nov. 12, 2009

(54) SHAFT-MOUNTED RF FILTERING ELEMENTS FOR IMPLANTABLE MEDICAL DEVICE LEAD TO REDUCE LEAD HEATING DURING MRI

(75) Inventor: Abhi Vase, San Jose, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/117,069

(22) Filed: May 8, 2008

(51) Int. Cl. A61N 1/00

(52) U.S. Cl. 607/37; 607/116

(57) ABSTRACT

Filtering components are provided for reducing heating within pacing/sensing leads of a pacemaker or other implantable medical device that occurs due to induced loop currents during magnetic resonance imaging (MRI) procedures. In one example, an inductive winding is provided around a non-conducting central portion of a shaft that interconnects a tip electrode of the lead to an inner coil conductor of the lead. By mounting the inductive winding to the shaft, inductive signal filtering can be readily provided so as to reduce tip heating, without requiring the incorporation of a lengthy, bulky inductor along the length of the lead. Capacitive elements may also be incorporated within the shaft to provide for LC filtering. In another example, the non-conducting central portion of the shaft is omitted. Instead, the conducting shaft end portions are interconnected by a stiff inductive winding, which functions as an air coil.

### FIELD OF THE INVENTION

The invention generally relates to leads for use with implantable medical devices, such as pacemakers or implantable cardioverter-defibrillators (ICDs) and, in particular, to configurations for installing radio-frequency (RF) filtering elements within such leads to reduce tip heating during magnetic resonance imaging (MRI) procedures.

### BACKGROUND OF THE INVENTION

MRI is an effective, non-invasive magnetic imaging technique for generating sharp images of the internal anatomy of the human body, which provides an efficient means for diagnosing disorders such as neurological and cardiac abnormalities and for spotting tumors and the like. Briefly, the patient is placed within the center of a large superconducting magnetic that generates a powerful static magnetic field. The static magnetic field causes protons within tissues of the body to align with an axis of the static field. A pulsed RF magnetic field is then applied causing the protons to begin to precess around the axis of the static field. Pulsed gradient magnetic fields are then applied to cause the protons within selected locations of the body to emit RF signals, which are detected by sensors of the MRI system. Based on the RF signals emitted by the protons, the MRI system then generates a precise image of the selected locations of the body, typically image slices of organs of interest.

However, MRI procedures are problematic for patients with implantable medical devices such as pacemakers and ICDs. One of the significant problems or risks is that the strong RF fields of the MRI can induce currents through the lead system of the implantable device into the tissues, resulting in Joule heating in the cardiac tissues around the electrodes of leads and potentially damaging adjacent tissues. Indeed, the temperature at the tip of an implanted lead has been found to increase as much as 70 degrees Celsius (C.) during an MRI tested in a gel phantom in a non-clinical configuration. Although such a dramatic increase is probably unlikely within a clinical system wherein leads are properly implanted, even a temperature increase of only about 8°-13° C. might cause myocardial tissue damage.

Furthermore, any significant heating of cardiac tissues near lead electrodes can affect the pacing and sensing parameters associated with the tissues near the electrode, thus potentially preventing pacing pulses from being properly captured within the heart of the patient and/or preventing intrinsic electrical events from being properly sensed by the device. The latter might result, depending upon the circumstances, in therapy being improperly delivered or improperly withheld. Another significant concern is that any currents induced in the lead system can potentially generate voltages within cardiac tissue comparable in amplitude and duration to stimulation pulses and hence might trigger unwanted contractions of heart tissue. The rate of such contractions can be extremely high, posing significant clinical risks to patients. Therefore, there is a need to reduce heating in the leads of implantable medical devices, especially pacemakers and ICDs, and to also reduce the risks of improper tissue stimulation during an MRI, which is referred to herein as MRI-induced pacing.

A variety of techniques have been developed to address these or other related concerns. See, for example, the following patents and patent applications: U.S. Pat. Nos. 6,871,091, 6,930,242, 6,944,489, 6,971,391, 6,985,775; U.S. Patent Application Nos. 2003/0083723, 2003/0083726, 2003/0144716, 2003/0144718, and 2003/0144719, and 2006/0085043; as well as the following PCT documents WO 03/037424, WO 03/063946, WO 03/063953. At least some of these techniques are directed to installing RF filters, such as inductive-capacitive (LC) filters, within the leads for use in filtering signals at frequencies associated with the RF fields of MRIs.

However, problems arise in the mounting of RF filters within medical device leads because the components are usually bulky. The size of a typical LC package is about 8 millimeters (mm) in length. This is particularly problematic since efforts are underway to reduce perforation and stiffness in the distal end of leads, efforts that are hindered by such bulky components. In addition, the incorporation of lengthy LC components can pose problems with the tip-ring spacing. Further, the presence of the LC-component raises issues concerning torque transfer within active fixation leads (i.e. leads that include a helical tip electrode for screw-in insertion into patient tissue to affix the lead).

Various aspects of the invention are directed to providing improved designs/structures for incorporating LC elements or other RF filtering elements into a lead, which address these and other concerns.

### SUMMARY OF THE INVENTION

In accordance with various exemplary embodiments of the invention, a lead is provided for use with an implantable medical device for implant within a patient wherein the lead includes an electrode for placement adjacent patient tissues, a conductor for routing signals along the lead and a shaft mounted between the conductor and the electrode. An inductive winding is mounted to the shaft and electrically connected to the electrode and the conductor. The inductive winding is configured to attenuate or filter high frequency electrical signals, particularly signals associated with current loops induced by the RF fields of MRI scans. By mounting the inductive winding to the shaft, rather than elsewhere within the lead, inductive filtering can be readily provided, without requiring the incorporation of a lengthy, bulky inductor along the length of the lead. Hence, problems pertaining to distal stiffness, torque transfer and tip-to- ring spacing are mitigated or eliminated. Also, the presence of the inductive element does not interfere with the extension or retraction of the tip electrode in active fixation leads. Capacitive elements may also be incorporated within the shaft so as to provide an LC element.

In a first illustrative embodiment, the shaft includes opposing conducting ends joined by a non-conducting central portion. The inductive winding is wound around the non-conducting central portion so as to form an inductor. The winding is preferably is covered with an insulating material such as a reflow material formed of, e.g., silicone polyurethane compound (SPC). Opposing ends of the winding are electrically connected to the conducting ends of the shaft, which are in turn connected to a tip electrode and an inner coil conductor, such as that inductive winding is in series with the tip electrode. In some examples, the non-conducting portion of the shaft is hollow and a capacitor is mounted therein. Opposing ends of the inductive winding and the capacitive element are electrically connected to one another to form an LC element for enhanced RF signal filtering.

In a second illustrative embodiment, two substantially coaxial shaft end portions are provided, with the inductive winding interconnecting the shafts and forming a chamber there-between. A proximal end of the inductive winding is electrically connected to a first, proximal shaft end portion, which is connected to the inner conducting coil for connection to the implantable device. A distal end of the inductive winding is electrically connected to a second, distal shaft end portion, which is connected to the tip electrode. The inductive winding thereby again provides an inductor connected in series with the tip electrode. The winding is also preferably covered with a reflow material such as SPC. In some examples, a capacitor is mounted within the chamber formed by the inductive winding. Opposing ends of the winding and the capacitive element are electrically connected to one another to form LC element. Titanium dioxide or other high-k dielectric material may be employed within the capacitive element.

In either embodiment, the electrical characteristics of the LC element are preferably selected so as to attenuate high frequency signals so as to prevent or reduce tip heating within the lead during an MRI procedure and to also prevent or reduce any MRI-induced pacing. Note that the pertinent frequencies to be filtered are the frequencies of currents induced in the media around the leads or inside leads, which are typically not the same as the frequencies of the MRI RF fields in air (which are typically 64 MHz or 128 MHz). The loops/signals to be filtered typically have wavelengths about equal to the length of the lead or integer multiples thereof.

These improved mounting designs/structures are particularly well suited for use with bipolar cardiac pacing/sensing leads for use with pacemakers and ICDs but may also be employed in connection with unipolar cardiac pacing/sensing leads or leads for use with other implantable medical devices. The leads may be passive fixation leads or active fixation leads.

### BRIEF DESCRIPTION OF THE DRAWINGS

The above and further features, advantages and benefits of the invention will be apparent upon consideration of the descriptions herein taken in conjunction with the accompanying drawings, in which:

FIG. 1 is a stylized representation of an MRI system along with a patient with a pacer/ICD implanted therein with RV and LV leads employing shaft-mounted RF filtering elements at their distal ends;

FIG. 2 is a block diagram, partly in schematic form, illustrating a bipolar lead for use with the pacer/ICD of FIG. 1 wherein a shaft-mounted RF filtering element is mounted between the tip electrode and tip conductor to reduce tip heating during an MRI, and also illustrating a pacer/ICD connected to the lead;

FIG. 3 is a side cross-sectional view of a portion of an active fixation embodiment of the lead of FIG. 2 shown with a helical tip electrode retracted, and particularly illustrating internal components of lead including the shaft-mounted RF filtering element;

FIG. 4 is a side elevational view of an alternative configuration of the shaft of FIG. 3;

FIG. 5 is a cross-sectional view of another alternative configuration of the shaft of FIG. 3, particularly illustrating the inductive winding and an internal capacitor;

FIG. 6 is a cross-sectional view of yet another alternative configuration of the shaft of FIG. 3, again illustrating the inductive winding and an internal capacitor;

FIG. 7 is a perspective view of just the inductor of FIG. 6;

FIG. 8 is a simplified, partly cutaway view, illustrating the pacer/ICD of FIG. 1 along with a more complete set of leads implanted in the heart of the patient, wherein the RV and LV leads include shaft-mounted RF filtering elements near tip electrodes of the leads; and

FIG. 9 is a functional block diagram of the pacer/ICD of FIG. 8, illustrating basic circuit elements that provide cardioversion, defibrillation and/or pacing stimulation in four chambers of the heart.

### DETAILED DESCRIPTION OF THE PREFERRED EMBODIMENTS

The following description includes the best mode presently contemplated for practicing the invention. The description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of MRI System

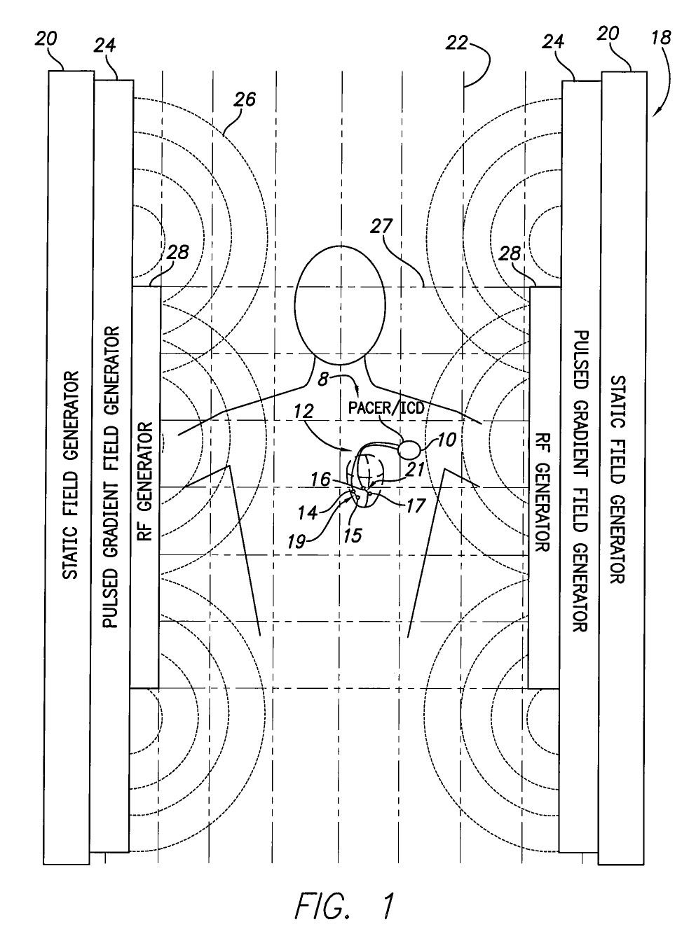

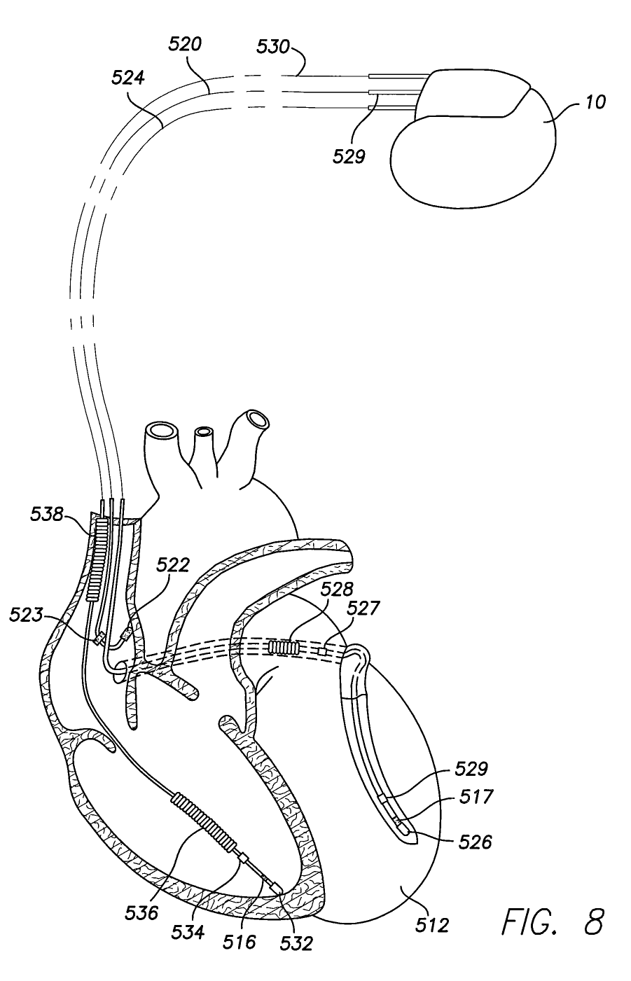

FIG. 1 illustrates an implantable medical system 8 having a pacer/ICD 10 for use with a set of coaxial bipolar pacing/sensing leads 12, which include tip and ring electrodes 14, 15, 16 and 17, as well as shaft-mounted RF filtering elements 19 and 21 (which are internal to the lead). The filtering elements are connected internally to the tip electrodes 15, 17 of the respective leads so as to reduce lead heating caused by loop currents generated by an MRI system 18 and to also reduce or prevent improper stimulation of the heart due to such loop currents. As will be explained further, the RF filtering elements are mounted to internal shafts (not shown in FIG. 1) near the distal ends of the leads. In FIG. 1, only two leads are shown, a right ventricular (RV) lead and a left ventricular (LV) lead. A more complete lead system is illustrated in FIG. 8, described below. In some implementations, one or more additional leads may be provided (such as a right atrial (RA) lead). RF filtering elements may be provided within the additional leads as well.

As to the MRI system 18, the MRI system includes a static field generator 20 for generating a static magnetic field 22 and a pulsed gradient field generator 24 for selectively generating pulsed gradient magnetic fields 26. The MRI system also includes an RF generator 28 for generating RF fields 27. Other components of the MRI, such as its sensing and imaging components are not shown. MRI systems and imaging techniques are well known and will not be described in detail herein. For exemplary MRI systems see, for example, U.S. Pat. No. 5,063,348 to Kuhara, et al., entitled “Magnetic Resonance Imaging System” and U.S. Pat. No. 4,746,864 to Satoh, entitled “Magnetic Resonance Imaging System.” Note that the fields shown in FIG. 1 are stylized representations of MRI fields intended merely to illustrate the presence of the fields. Actual MRI fields generally have far more complex patterns.

Hence, the leads of pacer/ICD 10 include RF filtering elements mounted to internal shafts and electrically connected to tip electrodes for use in reducing tip heating. The shaft-mounted filtering configurations described herein address the arrangement issues discussed above, such as problems relating to lead distal stiffness and tip-ring spacing. With reference to the remaining figures, the shaft-mounted RF filtering configurations will now be explained in greater detail.

### Shaft-Mounted RF Filter Arrangement Overview

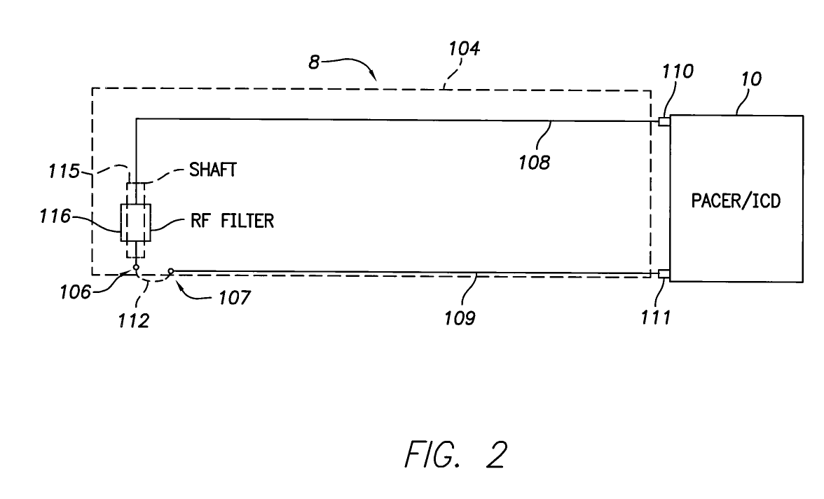

FIG. 2 illustrates an implantable system 8 having a pacer/ICD or other implantable medical device 10 with a bipolar coaxial lead 104. The bipolar lead includes a tip electrode 106 electrically connected to the pacer/ICD via a tip conductor 108 coupled to a tip connector or terminal 110 of the pacer/ICD. The bipolar lead also includes a ring electrode 107 electrically connected to the pacer/ICD via a ring conductor 109 coupled to a ring connector or terminal 111 of the pacer/ICD. Depending upon the particular implementation, during pacing/sensing, the tip electrode may be more negative than the ring, or vice versa. A conducting path 112 between tip electrode 106 and ring electrode 107 is provided through patient tissue (typically cardiac tissue.) An LC filtering element or other RF filter 116 is connected along conductor 108 at a distal portion thereof near tip electrode 106. The RF filter is mounted to a shaft 115, shown in phantom lines.

With the coaxial lead arrangement of FIG. 2, during an MRI, one or more current loops might be induced within the lead (and within any circuit components within the pacer/ICD that electrically connect terminals 110 and 111). The RF filter is configured to filter frequencies associated with such current loops to decrease the magnitude thereof. Without the RF filter, strong current loops might pass through patient tissue between the tip and ring electrodes before returning to the pacer/ICD, causing considerable resistive heating at the electrodes and in the intervening tissue. As explained above, such heating can damage patient tissue and interfere with pacing and sensing. In addition, as noted, the current loops can cause MRI-induced pacing. With RF filter 116, however, any such current loops are greatly diminished, thereby reducing a significant source of tip heating as well as preventing or limiting MRI-induced pacing.

The particular RF filter to be used may be chosen, at least in part, based on the frequency and magnitude of any current loops expected to be induced within the lead during an MRI, which may depend upon the location and orientation of the lead within the patient relative to the pacer/ICD and on the distance between the tip and ring electrodes and the impedance of tissues therebetween. Examples include L filters and LC filters. Different types of RF filters may be provided within atrial leads as compared to ventricular leads, with the filters of ventricular leads being generally more robust than the RF filters of the atrial leads since, typically, larger currents are induced in ventricular leads than in atrial leads during an MRI. Otherwise routine testing and experimentation may be performed to determine the appropriate parameters for the RF filter components for use in a particular lead for use in a particular patient so as to achieve adequate reduction in lead temperatures during an MRI within the patient or in the presence of other sources of strong RF fields.

### Shaft-Mounted LC Filter Examples

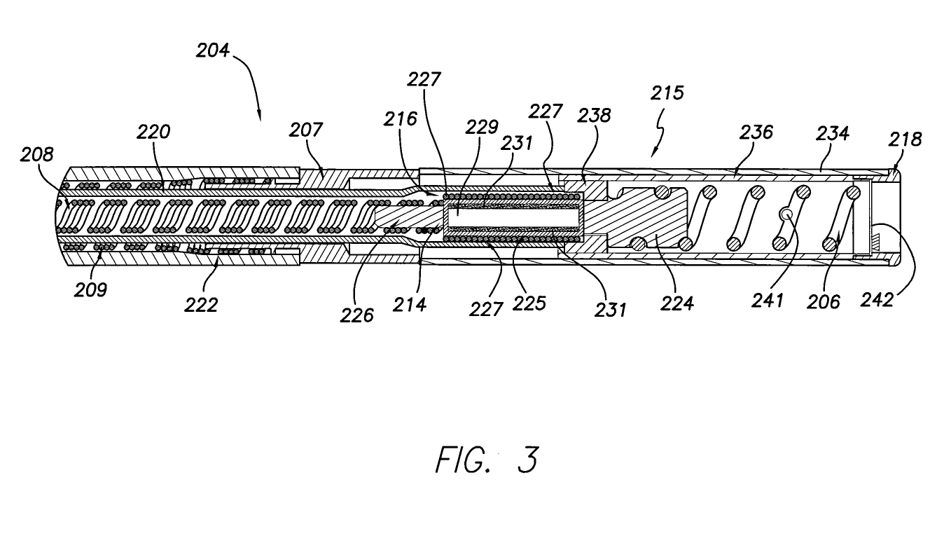

Turning now to FIGS. 3-7, exemplary configurations will be described in detail wherein the RF filter is an LC filtering element mounted to a shaft internal to the distal end of a bipolar cardiac pacing/sensing lead. Referring first to FIG. 3, an active fixation implementation of a lead 204 is shown. Lead 204 includes a helical tip electrode 206 coupled to an inner coil conductor 208 via an LC filter 216 mounted to shaft 214 inside a header 215. Helical tip 206 is shown in FIG. 3 in a retracted position within the header for mapping. While retracted, a mapping collar 218 is employed to map electrical characteristics of myocardial tissue to identify suitable locations for lead tip placement. (For non-mapping implementations, the mapping collar and the metal tubing connected thereto may be replaced with nonconducting polymer materials.) Once a suitable location is found, helical tip electrode 206 is extended to affix the distal end of the lead into the myocardial tissue. Thereafter, the helical tip electrode is used along with a ring electrode 207 to sense cardiac signals and to deliver pacing pulses. Ring electrode 207 is coupled to an outer coil conductor 209 for conducting return signals to the pacer/ICD via a proximal end of the lead (not shown in FIG. 3.)

Now considering the configuration of the lead in greater detail, the inner and outer coil conductors 208 and 209 are separated by insulation tubing 220. Outer insulation tubing 222 insulates the outer coil from patient tissues including blood. A proximal end of helical tip 206 is mounted to a metal distal end 224 of shaft 214, which is welded or bonded to non-conducting central portion 225 of shaft 214, which is in turn welded or bonded to metal proximal end 226 of shaft 214. Proximal end 226 of the shaft is fitted inside inner coil 208, as shown.

An inductive winding 227 is wrapped around central portion 225 of the shaft to function as an inductor. Although not specifically shown in FIG. 3, opposing ends of winding 227 are electrically connected to shaft ends 224 and 226, such that inductive winding is in series with the tip electrode along a current path extending from the implantable device through the tip electrode and into patient tissue. The central portion 225 of the shaft is, in this example, hollow so as to provide a chamber 229 in which a capacitor 231 is mounted, which is formed of a pair of plates. Although not specifically shown in FIG. 3, opposing proximal and distal terminals of capacitor 231 are electrically connected to the opposing ends of inductive winding 227 to collectively form LC element 216, wherein the inductive and capacitive elements are in parallel with one another between opposing ends 224 and 226 of the shaft. Electrical signals are routed between the tip terminal of the pacer/ICD (terminal 110 of FIG. 2) to tip electrode 206 via LC element 216, such that high frequency signals induced by MRI RF fields are significantly attenuated by the LC element, whereas low frequency signals associated with pacing, sensing and mapping are not significantly affected. Hence, with this configuration, the LC element functions to reduce tip heating and reduce the risk of any unwanted pacing pulses.

The various electrical connections (not shown in FIG. 3) to inductive coil 227 and to capacitor 231 can be achieved using any of a variety of techniques. For example, either a conductive metal or silicone can be used for connections or the two non-conducting sides of the shaft can be provided with features (not shown) to keep the inductor windings isolated. Direct welding can be used on the conducting sides. Another variation is to use insulative seals at either end of the winding with a welding tab providing a strong contact with the metal end-caps. The overall middle section or portion 225 of the shaft may be covered by a polytetrafluoroethylene (PTFE) sheath to isolate the inductor windings from SPC/silicone walls of central portion 225. Yet another variation utilizes an inductive winding reflowed with SPC, except at its ends. As such, the winding is electrically isolated from other components of the shaft and lead, with active ends not covered by reflow. In one particular example, the entire shaft is about 6 mm in length, with a central portion of about 3 mm in length. The diameter of the central portion is about 1.4 mm.

By mounting the inductive and capacitive components of LC element 216 to the shaft, rather than by mounting the LC element elsewhere between the tip and ring electrodes, the distance between the tip and ring electrodes need not be expanded to accommodate the LC element. In addition, with this shaft-mounted configuration, the presence of the LC element does not hinder the transference of torque, tension, and compression forces along the lead during implant/explant, including forces arising during extension or retraction of the helical tip within header 215.

Insofar as header 215 is concerned, an outer plastic insulating portion 234 encloses shaft 214 and the helical tip electrode 206, as shown. A conducting sheath 236 electrically connects mapping collar 218 to shaft end 224 via a metal spacer 238. To switch from the mapping configuration of the lead to the active fixation configuration, inner coil electrode 208 is twisted (via its proximal end, not shown), thereby causing the shaft to rotate, which in turn causes the helical tip electrode to rotate within the header. As the helical tip electrode rotates within the header, the helical electrode is fed past a post 241 and through a bracket 242 causing the distal end of the helical electrode to extend or protrude from the header into myocardial tissue in a screw-like manner. (In some examples, spacer 238 moves along with the shaft through the header while the tip electrode is extended. In other examples, the spacer is stationary but is configured so as to not block movement of the shaft.) Note that post 241 is small post built in the inside diameter surface of the header, which functions as a guider and stopper to guide the helix rotation and prevent over extension of the helix when extended. The post extends inwardly from an inner surface of sheath 236. In FIG. 3, the post is perpendicular to the cross-section slice and hence is only partially visible. If retraction of the helical electrode is subsequently required, the inner coil 208 is twisted in the opposite direction causing the shafts to rotate in the opposite sense, which in turn causes the helical tip electrode to retreat into the header. Mounting the LC element to the shaft helps ensure that the LC element does not interfere with extension/retraction of the helical tip electrode.

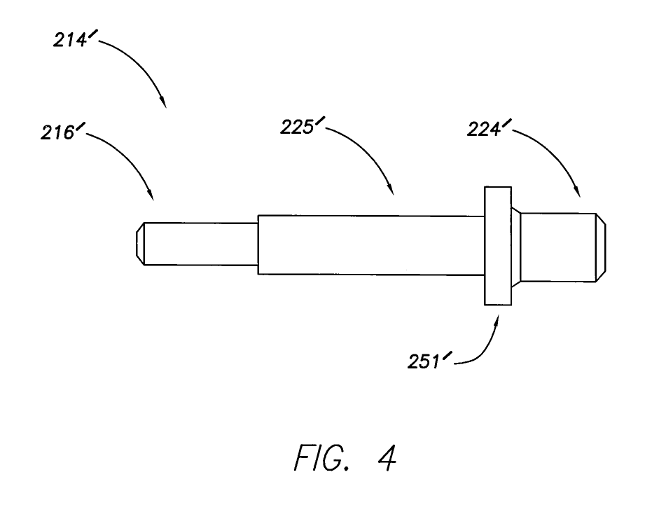

FIG. 4 illustrates an alternative shape for the shaft for use in a header (not shown) of differing internal construction. Shaft 214′ of FIG. 4 is assembled from three pieces: conducting distal and proximal ends 224′, 226′ and non-conducting central portion 225′. In this embodiment, distal end 224′ includes a protruding disk-shaped portion 251′, which helps position the shaft inside the header of the lead and limits its movement during tip extension/retraction. Distal and proximal ends 224′, 226′ are brazed to the central or middle portion 225′, which may be formed of glass or ceramic. Brazing may be achieved by coating the ends of the central portion with molybdenum/manganese and then using a filler braze metal.

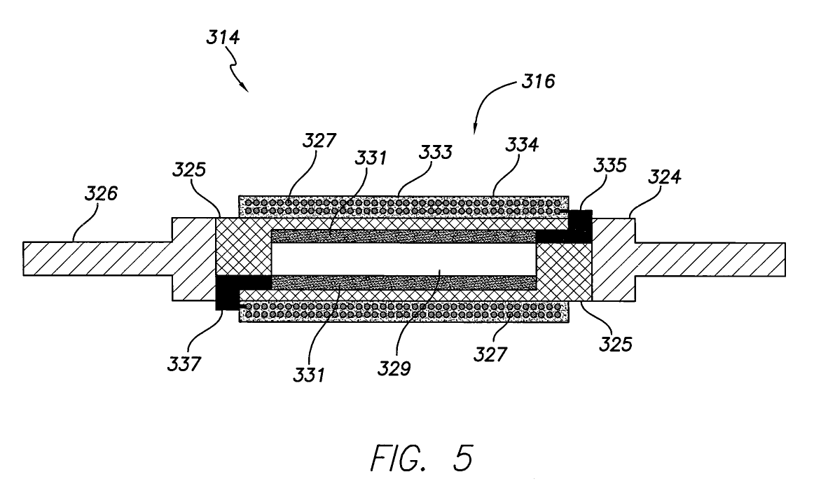

FIG. 5 illustrates an alternative configuration of the shaft and LC filter. As with the preceding embodiments, shaft 314 of FIG. 5 includes distal and proximal cylindrical ends 324, 326 connected by a central cylindrical conducting portion or section 325. Central portion 325 includes an air-filled chamber 329. The plates of a capacitor 331 are mounted to opposing surfaces of the central portion 325 of the shaft. Since the shaft is cylindrical, the plates are preferably curved so as to fit snugly against the inner surfaces of the shaft. An inductive winding 327 is wrapped around the central portion of the shaft. In this example, the winding is wrapped to have inner and outer rows or layers of coils, as shown. The windings are held within a reflow material 333 and collectively form an inductor 334. A distal electrical terminal 335 interconnects a distal end of winding 327 and a distal end of one of the two capacitor plates to distal shaft end 324. A proximal electrical terminal 337 interconnects a proximal end of winding 327 and a proximal end of the other of the two capacitor plates to proximal shaft end 326. As such, the inductor and the capacitor form an LC filter. The length of the central section is again about 3 mm with a diameter of 1.4 mm.

In one example, the LC filter is configured to provide 270 nanoHenries (nH) of inductance and 22 picoFarads (pF) of capacitance so as to significantly attenuate current loops induced by MRI scans operating at, e.g., 27 Mhz, 64 Mhz, or 128 MHz. The number of turns needed for the inductive winding may be calculated based on the frequency range of RF signals to be attenuated as follows. The basic inductance formula for a cylindrical coil may be represented by:

$$L = \frac{{μ}_{0} {μ}_{r} {N}^{2} A}{l}$$

where L=Inductance in henries (H);

μ₀=permeability of free space=4π×10⁻⁷ H/m;

μᵣ=relative permeability of core material;

N=number of turns;

A=area of cross-section of the coil in square meters (m²) and

l=length of coil in meters (m).

This equation may be solved for N given the desired inductance, the cross-sectional area and length of the coil, and the relative permeability of core material. Furthermore, using the following formulae, the number of turns can be calculated easily for American wire gauge (AWG) shafts. If the material changes, the number of turns likewise changes.

The inductance of a short air core cylindrical coil in terms of geometric parameters may be represented as:

$$L = \frac{{r}^{2} {N}^{2}}{9 r + 10 l}$$

where L=inductance in pH;

r=outer radius of coil in inches;

l=length of coil in inches; and

N=number of turns.

The inductance of a multilayer air core cylindrical coil in terms of geometric parameters may be represented as:

$$L = \frac{0.8 {r}^{2} {N}^{2}}{6 r + 9 l + 10 d}$$

where

L=inductance in pH;

r=mean radius of coil in inches;

l=physical length of coil winding in inches;

N=number of turns; and

d=depth of coil in inches (i.e., outer radius minus inner radius).

The inductance of an air core flat spiral coil in terms of geometric parameters may be represented as:

$$L = \frac{{r}^{2} {N}^{2}}{( 2 r + 2.8 d ) \times {10}^{5}}$$

where

L=inductance in H;

r=mean radius of coil in meters;

N=number of turns; and

d=depth of coil in meters (i.e., outer radius minus inner radius).

For the arrangement of FIG. 5, the air-core coil formula is the preferred formula to use, or one could instead use a formula for a non-metallic core and substitute the permittivity of that material. By way of example, calculations were performed for a multilayer air-core example with the dimensions driven by the shaft body dimensions (3 mm length and 1.4 mm diameter). The number of turns was in the range of 15-25, depending on material used and wire thickness. The number of layers (i.e. the concentric rows of windings) was in the range of 1-1.5. The overall thickness that the inductor adds to the shaft body may be, e.g., approximately 0.01″ (0.25 mm). Note that 36 AWG and 40 AWG embodiments are also suitable (the latter being preferred because good inductance properties have been observed with 3 mil gold bondwires for high frequency applications).

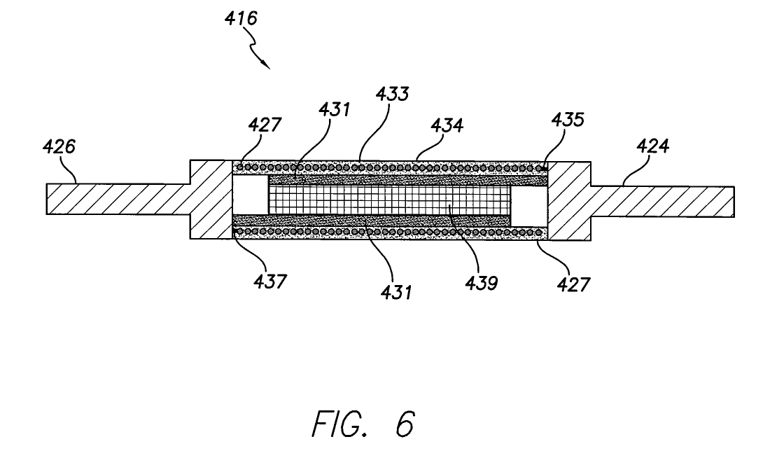

Insofar as the capacitors 231 are concerned, the dielectric between capacitor plates could be air (as shown in FIG. 5), but more preferably, the dielectric should be a high-k dielectric such as titanium dioxide. This is shown by way of the example of FIG. 6.

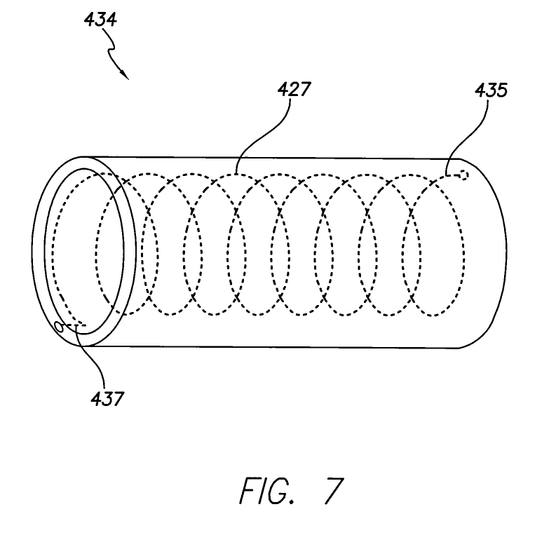

FIG. 6 illustrates an overall shaft 416 that includes distal and proximal shaft end portions 424, 426 connected by a cylindrical inductor 434 having an inductive winding 427 within a reflow material 433. In this example, the cylindrical inductor 434 forms the central portion of the overall shaft, which holds the opposing ends 424 and 426 together in a coaxial configuration. That is, the inductive windings are not wrapped around a separate center section material. Hence, rather than having an inductor wrapped around a central portion of a shaft, first and second shaft end portions 424 and 426 are provided, with inductor 434 interconnecting the shaft end portions so as to collectively form a single overall shaft structure. Inductor 434 is shown in FIG. 7 in a perspective view, with phantom lines schematically illustrating the internal windings 427.

As further shown in FIG. 6, inductor 434 forms a cylindrical chamber filled, at least partially, with titanium dioxide material 439 or other high-k dielectric. A capacitor 431 includes a pair of plates mounted to opposing surfaces of the dielectric, with each plate interposed between an outside surface of the dielectric 439 and an inside surface of inductor 434. The capacitor plates are preferably curved so as to fit snugly within the cylindrical inductor. A distal electrical terminal 435 interconnects a distal end of winding 427 to distal shaft end 424. A proximal electrical terminal 437 interconnects a proximal end of winding 427 to proximal shaft end 424. The capacitor plates are directly connected to shafts 424 and 426, as shown. The inductor and the capacitor again form an LC filter. The length of inductor 434 is about 3 mm with a diameter of 1.3 mm.

Insofar as capacitor 431 is concerned, the preferred capacitance value to be used depends on the dielectric material and other factors. Table I highlights the dependency on the dielectric for exemplary capacitors. In some examples, capacitances of about ˜22 pF have been found to be suitable.

TABLE I

| | Material | | | | |
|---|---|---|---|---|---|
| | TiO | | Ni—O | | Al—O |
| K | 80 | 80 | 40 | 40 | 28 |
| length {in} | 0.08 | 0.1 | 0.08 | 0.1 | 0.1 |
| width {in} | 0.02 | 0.02 | 0.02 | 0.02 | 0.02 |
| area {sq. in} | 0.0016 | 0.002 | 0.0016 | 0.002 | 0.002 |
| gap {in} | 0.001 | 0.001 | 0.001 | 0.001 | 0.001 |
| Cap | 28.7744 | 35.968 | 14.3872 | 17.984 | 12.5888 |

The various lead and shaft configurations described above can be exploited for use with a wide variety of implantable medical systems. In some embodiments, a micro-electro-mechanical systems (MEMS) device is mounted within the shaft and configured to filter signals associated with current loops induced by RF signals, as described in U.S. patent application Ser. No. 11/963243 of Vase et al., filed Dec. 21, 2007, entitled “MEMS-based RF Filtering Devices for Implantable Medical Device Leads to Reduce Lead Heating During MRI”.

For the sake of completeness, a detailed description of an exemplary pacer/ICD and lead system will now be provided.

### Exemplary Pacer/ICD/Lead System

FIG. 8 provides a simplified diagram of the pacer/ICD of FIG. 1, which is a dual-chamber stimulation device capable of treating both fast and slow arrhythmias with stimulation therapy, including cardioversion, defibrillation, and pacing stimulation. To provide atrial chamber pacing stimulation and sensing, pacer/ICD 10 is shown in electrical communication with a heart 512 by way of a left atrial lead 520 having an atrial tip electrode 522 and an atrial ring electrode 523 implanted in the atrial appendage. Pacer/ICD 10 is also in electrical communication with the heart by way of a right ventricular lead 530 having, in this embodiment, a ventricular tip electrode 532, a right ventricular ring electrode 534, a right ventricular (RV) coil electrode 536. Typically, the right ventricular lead 530 is transvenously inserted into the heart so as to place the RV coil electrode 536 in the right ventricular apex. Accordingly, the right ventricular lead is capable of receiving cardiac signals, and delivering stimulation in the form of pacing and shock therapy to the right ventricle. A shaft-mounted LC filtering element 516, configured as described above, is positioned within a distal end of lead 530 near tip electrode 532 for use in attenuating high frequency signals so as to reduce lead heating. In the figure, the shaft-mounted LC filtering element is shown in phantom lines, as it is internal to the lead.

To sense left atrial and ventricular cardiac signals and to provide left chamber pacing therapy, pacer/ICD 10 is coupled to a “coronary sinus” lead 524 designed for placement in the “coronary sinus region” via the coronary sinus os for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, an exemplary coronary sinus lead 524 is designed to receive atrial and ventricular cardiac signals and to deliver left ventricular pacing therapy using at least a left ventricular tip electrode 526 and a left ventricular ring electrode 529 and to deliver left atrial pacing therapy using at least a left atrial ring electrode 527, and shocking therapy using at least an SVC coil electrode 528. A shaft-mounted LC filtering element 517, configured as described above, is positioned within a distal end of lead 524 near tip electrode 526 for use in attenuating high frequency signals so as to reduce lead heating. Again, the LC filtering element is shown in phantom lines, as it is internal to the lead. Although not shown, shaft-mounted LC filtering elements may also be provided within RA lead 520.

With this configuration, biventricular pacing can be performed. Although only three leads are shown in FIG. 8, it should also be understood that additional stimulation leads (with one or more pacing, sensing and/or shocking electrodes) may be used in order to efficiently and effectively provide pacing stimulation to the left side of the heart or atrial cardioversion and/or defibrillation.

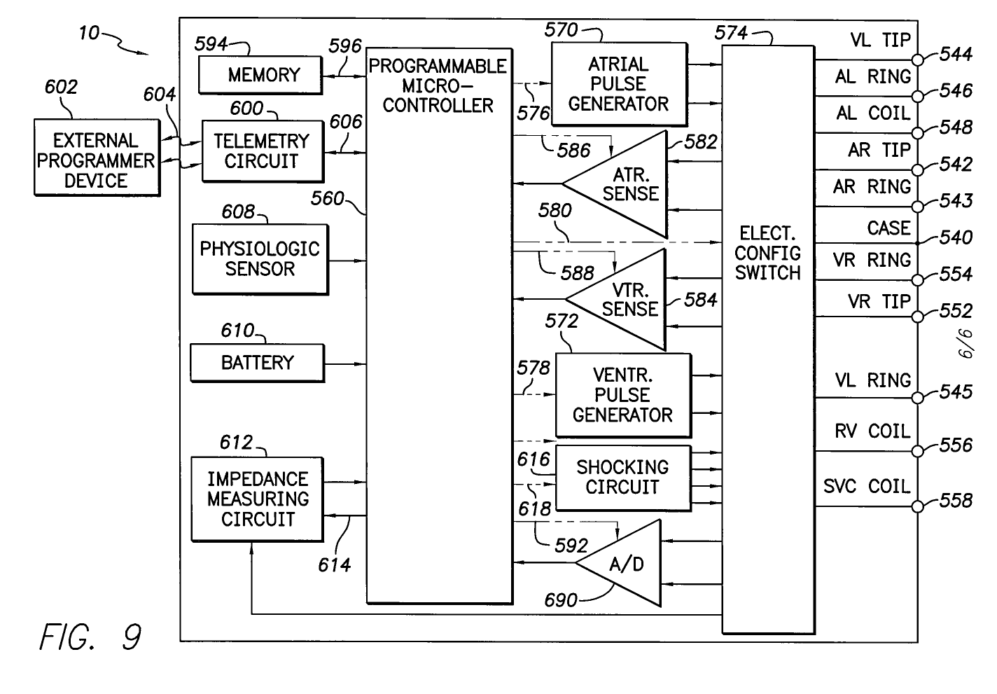

A simplified block diagram of internal components of pacer/ICD 10 is shown in FIG. 9. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation as well as providing for the aforementioned apnea detection and therapy.

The housing 540 for pacer/ICD 10, shown schematically in FIG. 9, is often referred to as the “can”, “case” or “case electrode” and may be programmably selected to act as the return electrode for all “unipolar” modes. The housing 540 may further be used as a return electrode alone or in combination with one or more of the coil electrodes, 528, 536 and 538, for shocking purposes. The housing 540 further includes a connector (not shown) having a plurality of terminals, 542, 543, 544, 545, 546, 548, 552, 554, 556 and 558 (shown schematically and, for convenience, the names of the electrodes to which they are connected are shown next to the terminals). As such, to achieve right atrial sensing and pacing, the connector includes at least a right atrial tip terminal (A_{R} TIP) 542 adapted for connection to the atrial tip electrode 522 and a right atrial ring (A_{R} RING) electrode 543 adapted for connection to right atrial ring electrode 523. To achieve left chamber sensing, pacing and shocking, the connector includes at least a left ventricular tip terminal (V_{L} TIP) 544, a left ventricular ring terminal (V_{L} RING) 545, a left atrial ring terminal (A_{L} RING) 546, and a left atrial shocking terminal (A_{L} COIL) 548, which are adapted for connection to the left ventricular ring electrode 526, the left atrial tip electrode 527, and the left atrial coil electrode 528, respectively. To support right chamber sensing, pacing and shocking, the connector further includes a right ventricular tip terminal (V_{R} TIP) 552, a right ventricular ring terminal (V_{R} RING) 554, a right ventricular shocking terminal (R_{V} COIL) 556, and an SVC shocking terminal (SVC COIL) 558, which are adapted for connection to the right ventricular tip electrode 532, right ventricular ring electrode 534, the RV coil electrode 536, and the SVC coil electrode 538, respectively.

At the core of pacer/ICD 10 is a programmable microcontroller 560, which controls the various modes of stimulation therapy. As is well known in the art, the microcontroller 560 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 560 includes the ability to process or monitor input signals (data) as controlled by a program code stored in a designated block of memory. The details of the design and operation of the microcontroller 560 are not critical to the invention. Rather, any suitable microcontroller 560 may be used that carries out the functions described herein. The use of microprocessor-based control circuits for performing timing and data analysis functions are well known in the art.

As shown in FIG. 9, an atrial pulse generator 570 and a ventricular pulse generator 572 generate pacing stimulation pulses for delivery by the right atrial lead 520, the right ventricular lead 530, and/or the coronary sinus lead 524 via an electrode configuration switch 574. It is understood that in order to provide stimulation therapy in each of the four chambers of the heart, the atrial and ventricular pulse generators, 570 and 572, may include dedicated, independent pulse generators, multiplexed pulse generators or shared pulse generators The pulse generators, 570 and 572, are controlled by the microcontroller 560 via appropriate control signals, 576 and 578, respectively, to trigger or inhibit the stimulation pulses.

The microcontroller 560 further includes timing control circuitry (not separately shown) used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 574 includes a plurality of switches for connecting the desired electrodes to the appropriate I/O circuits, thereby providing complete electrode programmability. Accordingly, the switch 574, in response to a control signal 580 from the microcontroller 560, determines the polarity of the stimulation pulses (e.g., unipolar, bipolar, combipolar, etc.) by selectively closing the appropriate combination of switches (not shown) as is known in the art.

Atrial sensing circuits 582 and ventricular sensing circuits 584 may also be selectively coupled to the right atrial lead 520, coronary sinus lead 524, and the right ventricular lead 530, through the switch 574 for detecting the presence of cardiac activity in each of the four chambers of the heart. Accordingly, the atrial (ATR. SENSE) and ventricular (VTR. SENSE) sensing circuits, 582 and 584, may include dedicated sense amplifiers, multiplexed amplifiers or shared amplifiers. The switch 574 determines the “sensing polarity” of the cardiac signal by selectively closing the appropriate switches, as is also known in the art. In this way, the clinician may program the sensing polarity independent of the stimulation polarity. Each sensing circuit, 582 and 584, preferably employs one or more low power, precision amplifiers with programmable gain and/or automatic gain control and/or automatic sensitivity control, bandpass filtering, and a threshold detection circuit, as known in the art, to selectively sense the cardiac signal of interest. The automatic gain and/or sensitivity control enables pacer/ICD 10 to deal effectively with the difficult problem of sensing the low amplitude signal characteristics of atrial or ventricular fibrillation. The outputs of the atrial and ventricular sensing circuits, 582 and 584, are connected to the microcontroller 560 which, in turn, are able to trigger or inhibit the atrial and ventricular pulse generators, 570 and 572, respectively, in a demand fashion in response to the absence or presence of cardiac activity in the appropriate chambers of the heart.

For arrhythmia detection, pacer/ICD 10 utilizes the atrial and ventricular sensing circuits, 582 and 584, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation which are sometimes referred to as “Fib-waves”) are then classified by the microcontroller 560 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 590. The data acquisition system 590 is configured to acquire intracardiac electrogram signals, convert the raw analog data into a digital signal, and store the digital signals for later processing and/or telemetric transmission to an external device 602. The data acquisition system 590 is coupled to the right atrial lead 520, the coronary sinus lead 524, and the right ventricular lead 530 through the switch 574 to sample cardiac signals across any pair of desired electrodes. The microcontroller 560 is further coupled to a memory 594 by a suitable data/address bus 596, wherein the programmable operating parameters used by the microcontroller 560 are stored and modified, as required, in order to customize the operation of pacer/ICD 10 to suit the needs of a particular patient. Such operating parameters define, for example, pacing pulse amplitude or magnitude, pulse duration, electrode polarity, rate, sensitivity, automatic features, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 10 may be non-invasively programmed into the memory 594 through a telemetry circuit 600 in telemetric communication with an external device 602, such as a programmer, transtelephonic transceiver or a diagnostic system analyzer, or a bedside monitoring system. The telemetry circuit 600 is activated by the microcontroller by a control signal 606. The telemetry circuit 600 advantageously allows IEGMs and other electrophysiological signals and/or hemodynamic signals and status information relating to the operation of pacer/lCD 10 (as stored in the microcontroller 560 or memory 594) to be sent to the external programmer device 602 through an established communication link 604.

Pacer/ICD 10 further includes an accelerometer or other physiologic sensor 608, commonly referred to as a “rate-responsive” sensor because it is typically used to adjust pacing stimulation rate according to the exercise state of the patient. However, the physiological sensor 608 may further be used to detect changes in cardiac output, changes in the physiological condition of the heart, or diurnal changes in activity (e.g., detecting sleep and wake states) and to detect arousal from sleep. Accordingly, the microcontroller 560 responds by adjusting the various pacing parameters (such as rate, AV Delay, V-V Delay, etc.) at which the atrial and ventricular pulse generators, 570 and 572, generate stimulation pulses. While shown as being included within pacer/ICD 10, it is to be understood that the physiologic sensor 608 may also be external to pacer/lCD 10, yet still be implanted within or carried by the patient. A common type of rate responsive sensor is an activity sensor incorporating an accelerometer or a piezoelectric crystal, which is mounted within the housing 540 of pacer/ICD 10. Other types of physiologic sensors are also known, for example, sensors that sense the oxygen content of blood, respiration rate and/or minute ventilation, pH of blood, ventricular gradient, etc.

The pacer/ICD additionally includes a battery 610, which provides operating power to all of the circuits shown in FIG. 9. The battery 610 may vary depending on the capabilities of pacer/ICD 10. If the system only provides low voltage therapy, a lithium iodine or lithium copper fluoride cell may be utilized. For pacer/ICD 10, which employs shocking therapy, the battery 610 must be capable of operating at low current drains for long periods, and then be capable of providing high-current pulses (for capacitor charging) when the patient requires a shock pulse. The battery 610 must also have a predictable discharge characteristic so that elective replacement time can be detected. Accordingly, pacer/ICD 10 is preferably capable of high voltage therapy and appropriate batteries.

As further shown in FIG. 9, pacer/ICD 10 is shown as having an impedance measuring circuit 612 which is enabled by the microcontroller 560 via a control signal 614. Various uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, measuring lead resistance, etc. The impedance measuring circuit 120 is advantageously coupled to the switch 64 so that any desired electrode may be used.

In the case where pacer/ICD 10 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 560 further controls a shocking circuit 616 by way of a control signal 618. The shocking circuit 616 generates shocking pulses of low (up to 0.5 joules), moderate (0.5-11 joules) or high energy (11 to at least 40 joules), as controlled by the microcontroller 560. Such shocking pulses are applied to the heart of the patient through at least two shocking electrodes, and as shown in //this embodiment, selected from the left atrial coil electrode 528, the RV coil electrode 536, and/or the SVC coil electrode 538. The housing 540 may act as an active electrode in combination with the RV electrode 536, or as part of a split electrical vector using the SVC coil electrode 538 or the left atrial coil electrode 528 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 11-40 joules), delivered asynchronously (since R-waves may be too disorganized), and pertaining exclusively to the treatment of fibrillation. Accordingly, the microcontroller 560 is capable of controlling the synchronous or asynchronous delivery of the shocking pulses.

What have been described are systems and methods for use with a set of pacing/sensing leads for use with a pacer/ICD. Principles of the invention may be exploiting using other implantable systems or in accordance with other techniques. Thus, while the invention has been described with reference to particular exemplary embodiments, modifications can be made thereto without departing from the scope of the invention.

What is claimed is:

1. A lead for use with an implantable medical device for implant within a patient, the lead comprising:
an electrode for placement adjacent patient tissues;
a conductor for routing signals along the lead;
a shaft mounted between the conductor and the electrode; and
an inductive winding mounted to the shaft, the winding electrically connecting the electrode and the conductor.

2. The lead of claim 1 wherein the shaft includes a non-conducting portion and wherein the inductive winding is wound around the non-conducting portion.

3. The lead of claim 2 wherein the non-conducting portion of the shaft is a central portion of the shaft and wherein opposing end portions of the shaft are conducting.

4. The lead of claim 2 wherein the inductive winding is covered with an insulating material.

5. The lead of claim 4 wherein the insulating material is a silicone polyurethane compound (SPC).

6. The lead of claim 2 further including a capacitive element positioned within the non-conducting portion of the shaft, the inductive winding and the capacitive element electrically connected to one another at opposing ends to form an inductive-capacitive (LC) element.

7. The lead of claim 6 wherein distal terminals of the inductive winding and the capacitive element are electrically connected to the electrode and wherein proximal terminals of the inductive winding and the capacitive element are electrically connected to the conductor so that the inductive winding and the capacitive element are connected in parallel within one another.

8. The lead of claim 1 wherein a pair of substantially coaxial shaft end portions are provided, with the inductive winding interconnecting the shaft end portions, the inductive winding forming a chamber between the pair of shaft end portions.

9. The lead of claim 8 wherein the chamber formed by the inductive winding is filled with air.

10. The lead of claim 8 wherein each of the pair of shaft end portions is conducting.

11. The lead of claim 10 wherein a proximal end of the inductive winding is electrically connected to a first, proximal shaft end portion and wherein a distal end of the inductive winding is electrically connected to a second, distal shaft end portion.

12. The lead of claim 11 wherein the inductive winding is covered with an insulating material.

13. The lead of claim 12 wherein the insulating material is a silicone polyurethane compound (SPC).

14. The lead of claim 12 further including a capacitive element positioned within an inner surface of the inductive winding, with the inductive winding and the capacitive element electrically connected to one another at opposing ends to form an inductive-capacitive (LC) element.

15. The lead of claim 14 wherein distal terminals of the inductive winding and the capacitive element are electrically connected to the distal shaft end portion and wherein proximal terminals of the inductive winding and the capacitive element are electrically connected to the proximal shaft end portion so that the inductive winding and the capacitive element are connected in parallel within one another.

16. The lead of claim 14 further including a dielectric material positioned within the capacitive element.

17. The lead of claim 16 wherein the dielectric material includes titanium dioxide.

18. An inductive element for use within a lead of an implantable medical device for implant within a patient, the inductive element for electrical connection between an electrode for placement adjacent patient tissues and a conductor for routing signals along the lead, the inductive element comprising:
a shaft having a pair of conducting end portions for connection, respectively, to the conductor and the electrode; and
an inductive winding mounted to the shaft, the winding electrically connecting the electrode and the conductor.

19. An implantable medical system for implant within a patient comprising:
an implantable cardiac rhythm management device; and
a lead for use with the implantable medical device wherein the lead includes an electrode for placement adjacent patient tissues;
conductor for routing signals along the lead; a shaft mounted between the conductor and the electrode; and an inductive winding mounted to the shaft, the winding electrically connecting the electrode and the conductor of the lead.
